(19) United States
(12) Patent Application Publication
(10) Pub. No.: US 2022/0384026 A1
(43) Pub. Date: Dec. 1, 2022

(54) PATIENT DOCTOR INTERACTION SYSTEM, MEDICAL QUICK RESPONSE CODE SYSTEM, DOCTOR PATIENT DIAGNOSIS SHARING INFORMATION SYSTEM, DOCTOR PATIENT COMMUNICATION SYSTEM, PROCESS, AND METHOD OF USE

(71) Applicants: Ayman H. Al-Jazaeri, Riyadh (SA); Ahmad H. Al-Jazaeri, Riyadh (SA)

(72) Inventors: Ayman H. Al-Jazaeri, Riyadh (SA); Ahmad H. Al-Jazaeri, Riyadh (SA)

(21) Appl. No.: 17/751,717

(22) Filed: May 24, 2022

Related U.S. Application Data
(60) Provisional application No. 63/192,596, filed on May 25, 2021.

(51) Int. Cl. G16H 40/63; G06K 19/06; G06K 7/14; G16H 20/10

(52) U.S. Cl. CPC G16H 40/63; G06K 19/06037; G06K 7/1417; G16H 20/10

(57) ABSTRACT

A patient doctor interaction system, medical quick response code system, doctor patient diagnosis information sharing system, doctor patient communication system, process, and methods of use are presented. The present disclosure provides an interaction which provides a patient with information unique to their ailment and their circumstances, an ability for ongoing communication, if necessary, after a patient leaves the office of a practitioner and after the medical interview has concluded, a diagnosis and relay of complex medical information to the client that is not limited to a verbal, short interaction in which information may be rushed or delivered in a way the patient does not understand, and a system in which a patient is able to interact with the practitioner without being present in person and without requiring a presence in person or a phone call, which may be difficult to get a hold of the practitioner.

### CROSS-REFERENCE TO RELATED APPLICATIONS

The present application claims priority to the U.S. Patent Application No. 63/192,596 which was filed on May 25, 2022, which is hereby incorporated by reference herein in its entirety, including any figures, tables, or drawings.

### FIELD OF THE DISCLOSURE

This disclosure relates to a patient doctor interaction system. More specifically, and without limitation, the present disclosure relates to a system for providing information sharing between a service receiver and/or patient and a service provider and/or doctor related to a diagnosis and/or treatment plan. More specifically, and without limitation, the present disclosure relates to a doctor patient communication system, quick sharing and/or identification system, process, and methods of use.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright. Ayman Al-Jazaeri. All rights reserved.

### BACKGROUND OF THE DISCLOSURE

A trained physician providing patient care is well known in the art. A practitioner providing care to a patient may also be referred to as a doctor-patient relationship. This relationship is very complex. This relationship is formed when a patient experiences an ailment or wants health maintenance services and seeks consultation and the expertise of a practitioner or doctor. The relationship often involves at least one meeting, may involve complex matters of trust, communication and understanding of the parties involved and may sometimes include a relationship built over a period of many years. Various complexities of these interactions are shaped through doctors' oaths as practitioners, legal aspects such as laws, ranging from privacy laws to liability laws. This relationship is a type of relationship that exists across many cultures and has existed for many millennia, experiencing few changes over those millennia.

This doctor-patient care and relationship is very important in overall healthcare. Even with the importance of doctor-patient care, doctor-patient communication, and doctor-patient interactions, little has changed in this interaction and in these communications since the inception of consultant-patient care. Oftentimes the delivery of patient care can be a matter of life and death. Thus, the doctor-patient care relationship is critical in human health and well-being. Still little improvements have been made in this art, and various environmental factors such as the Covid 19 pandemic have affected this relationship.

Furthermore, the medium in which data is shared, data is gathered, diagnosis are made, health treatment plans are conveyed, compliance is accomplished, healing is monitored, patient activation is handled, and the means through which support is provided have remained unchanged and unaltered and unimproved. Due to complexities in treatment and more, it is very difficult to make changes and/or improvements to this state of the art.

A typical doctor-patient interaction may go as follows. A patient experiences an ailment. A patient may make an appointment with a new doctor or an established doctor which has records of the patient's health history, or partial history, or no history at all. In some circumstances a patient may have an ailment which requires a visit to an emergency room for immediate care, this care is likely from a practitioner which is unfamiliar with the patient and/or the patient's healthcare.

During this patient's visit to a practitioner, a medical interview will take place. Medical interviews are very important in modern healthcare and the person to person interaction of the medical interview is a well known medium of healthcare. Most medical encounters are spent in this medical interview, being a discussion about the ailment or ailments, between the practitioner and the patient. The medical interview has three major functions, which are well known in the art. The three major functions are 1) the gathering of information, 2) the development and maintenance of the practitioner-patient relationship, and 3) the communication of information. Furthermore, the medical interaction may be a direct interaction, a verbal interaction, or similar interaction through phone or teleconference, and the like. Furthermore, the interaction may also include formulating diagnosis and treatment plans and conveying the plan to the patient in verbal and/or written forms.

The purpose of the medical interview, the gathering of information, development of the relation, and the communication of information is so that the practitioner can learn about the patient, the ailments, and create a plan to improve the life of the patient which often involves a diagnosis and treatment. This is most commonly the next step in a typical doctor-patient interaction. The practitioner, once they believe they understand the ailment, will prepare a diagnosis and treatment plan. Once the plan is created, the doctor or practitioner will attempt to communicate an understanding of that ailment and the treatment plan created.

Following the interaction, if a medication or prescription plan and/or instruction slip is created, which may include obtaining medicines by prescription and/or instruction, over the counter medicine, physical exercises, applications of ice, heat and the like, or other information which can aid in the treatment of the ailment or ailments. In some rare instances, a representative or the practitioner themselves will follow up to see if the treatment or medicine has worked in solving the ailment. In other situations, the ailment may persist and another visit to the same practitioner or another practitioner may be required to further define the ailment and come up with further treatment.

Thus, there is a long-felt need in the art for an improvement to the practitioner-patient interaction and relationship. Furthermore, the demands on healthcare providers today are extreme and tend to be high. For this reason, healthcare providers' time is very valuable. Therefore, anything that can save time for a healthcare provider is valuable. Thus, the present disclosure, and practices of the present disclosure, save time for a healthcare provider and add value to the work of a healthcare provider. Furthermore, the present disclosure improves the efficiency of work and interactions which permits a healthcare provider to offer expert services to a larger group and in a better way. There is a need in the art for an interaction which provides a patient with information unique to their ailment and their circumstances. Furthermore, there is a need in the art for an ability for ongoing communication, if necessary, after a patient leaves the office of a practitioner and after the medical interview, telemedicine interview and/or video conferencing has concluded. Furthermore, there is a need in the art for a diagnosis and relay of complex medical information to the client that is not limited to a verbal, short interaction in which information may be rushed or delivered in a way the patient does not understand. Furthermore, there is a need in the art for a system in which a patient is able to interact with the practitioner without being present in person and without requiring a presence in person or a phone call, which may be difficult to get a hold of the practitioner. Additionally, the present disclosure provides access to tools which enhance a provider's work through efficiency and accuracy. Said another way, the present disclosure provides technology which provides for increased and improved accuracy in a provider's work. This enhances details through audio-visual tools and the like. Furthermore, the present disclosure provides the ability for a patient and others to recall the instructions previously provided. This improves upon health of patients, users, and the like.

The present disclosure provides these advantages and others as will become clear from the specification and claims provided.

### SUMMARY OF THE DISCLOSURE

A patient doctor interaction system is presented. More specifically, and without limitation, the present disclosure relates to a system for providing information sharing between a patient and a doctor related to a diagnosis and/or treatment plan. More specifically, and without limitation, the present disclosure relates to a doctor patient communication system, quick sharing and/or identification system, process, and methods of use.

More specifically, and without limitation, the present disclosure provides the state of the art with a medical quick response code portal system for use between a practitioner and a client. The present disclosure also includes a particular process for sharing information and a method of using the system disclosed herein.

The present disclosure provides the state of the art with and includes as part of the system, a quick response code display. The quick response display may be on a sheet of paper, a smartphone, smart device, tablet, smart watch, or computer, or other display screen such as a computer screen or television or the like. In the present disclosure the quick response code display shows a plurality of quick response codes for the purpose of scanning these quick response codes.

In the present disclosure, each of the plurality of quick codes is representative of and relates to a discrete patient conditions and/or patient ailment and/or patient treatment. Furthermore, each of the plurality of quick response codes may relate to a customized and particular treatment or the like which is a particular physician's plan for a patient, a particular physician group's plan for a patient, or a particular hospital's plan for a patient. Furthermore, each of the plurality of quick response codes contains information for web addresses and other information related to a landing page containing patient information and/or instructions for a particular condition and/or treatment.

In the present disclosure, during a clinical visit—whether it be at a clinic, hospital, home, pop-up location or the like—with a patient (via in-person, audio, video, or a combination thereof), a physician will identify one or more quick response codes of the plurality of quick response codes within the quick response code display. The quick response code identified by the physician will be the relevant code to and/or appropriate to the particular patient's condition and/or treatment.

In the present disclosure, the physician will have the patient scan the relevant quick code or quick codes with a quick response reader and/or quick response scanner on the patient's device (such as a mobile device or table). By scanning the selected quick code or quick codes, a landing page containing patient information—being the provider's instructions—is loaded to the device of the patient. The patient can then refer to this page even after the interaction with the physician is complete. This provides the patient with additional and ongoing availability of information. This enables the patient to stay informed and to be able to continue effective treatment without reliance on memory and the like. Said another way, this provides the patient with ongoing access to instructions appropriate for the patient for the particular condition and/or particular treatments needed for the particular patient. In the arrangement shown, as one example, the provider creates unique information pertaining to the patient and provides this information to the patient, as opposed to the patient being only supplied with generic information.

In the present disclosure, the medical quick response code portal system and the medical quick response code method of use also includes back end software which enables the functionality herein as well as graphical user interfaces for both the physician and the patient which enable these various interactions. Furthermore, this system enables staff of the physician and others to customize the information and/or customize the instructions for a particular patient, and/or a particular treatment and/or particular conditions.

Furthermore, the present disclosure also includes a paper option. In this embodiment the quick response code display may be a prescription booklet and/or printed and/or in the form of a billboard and/or displayed on a public screen and/or monitor and/or smart device, so as to display the plurality of quick response codes on one or more pages of a booklet. The present disclosure also provides a means of providing this plurality of quick response codes to a user through electronic delivery such as through email or text message and the like. In this way, the present disclosure of quick response codes can be delivered to a patient and/or patients without needing to see the patient in person. In this way, the present disclosure provides for a system of a meeting between a physician and a patient virtually, a diagnosis to be provided virtually, and the various quick code responses to be provided in a unique way which suits the patient as well as are customized for the patient.

Thus, it is a primary object of the disclosure to provide a patient doctor interaction system, medical quick response code system, doctor patient diagnosis information sharing system, doctor patient communication system, process, and methods of use that improve upon the state of the art or provide unique systems and methods of using these systems which have not been used in the art before, and are thus novel and nonobvious for the state of the art.

Another object of the disclosure is to provide a patient doctor interaction system, medical quick response code system, doctor patient diagnosis information sharing system, doctor patient communication system, process, and methods of use that utilizes quick response codes.

Yet another object of the disclosure is to provide a patient doctor interaction system, medical quick response code system, doctor patient diagnosis information sharing system, doctor patient communication system, process, and methods of use that provides a patient with access to explanations and/or information about ailment.

Another object of the disclosure is to provide a patient doctor interaction system, medical quick response code system, doctor patient diagnosis information sharing system, doctor patient communication system, process, and methods of use that provides a patient with ongoing access to information about the patients treatment; such as doctor and/or provider specific information, information being easily accessible regardless of time and place of the provider-patient interaction.

Yet another object of the disclosure is to provide a patient doctor interaction system, medical quick response code system, doctor patient diagnosis information sharing system, doctor patient communication system, process, and methods of use that provides a patient with ongoing communication with a physician and/or doctor and/or provider.

Another object of the disclosure is to provide a patient doctor interaction system, medical quick response code system, doctor patient diagnosis information sharing system, doctor patient communication system, process, and methods of use that provides a quick response code to a patient that corresponds to the patient's specific illness.

Yet another object of the disclosure is to provide a patient doctor interaction system, medical quick response code system, doctor patient diagnosis information sharing system, doctor patient communication system, process, and methods of use that provides a quick response code to a patient that corresponds to the patient's specific treatment plan.

Another object of the disclosure is to provide a patient doctor interaction system, medical quick response code system, doctor patient diagnosis information sharing system, doctor patient communication system, process, and methods of use that provides a patient with access to an instruction page customized by the physician with treatment for the patient, and other information customized for the patient.

Yet another object of the disclosure is to provide a patient doctor interaction system, medical quick response code system, doctor patient diagnosis information sharing system, doctor patient communication system, process, and methods of use that provide a unique doctor patient interaction.

Another object of the disclosure is to provide a patient doctor interaction system, medical quick response code system, doctor patient diagnosis information sharing system, doctor patient communication system, process, and methods of use that allows a patient to share and/or save a topic for access at a later time; also share a topic and/or information with others.

Yet another object of the disclosure is to provide a patient doctor interaction system, medical quick response code system, doctor patient diagnosis information sharing system, doctor patient communication system, process, and methods of use that provide various statistics and quantitative feedback about a doctors performance with regards to doctor patient interactions.

Another object of the disclosure is to provide a patient doctor interaction system, medical quick response code system, doctor patient diagnosis information sharing system, doctor patient communication system, process, and methods of use that provides a patient with ongoing access to text, instruction media, and photos and videos.

Yet another object of the disclosure is to provide a patient doctor interaction system, medical quick response code system, doctor patient diagnosis information sharing system, doctor patient communication system, process, and methods of use that allows a doctor to edit and update patient treatments and information made available to a patient even after a medical interview and/or other virtual meeting, and/or the like.

Another object of the disclosure is to provide a patient doctor interaction system, medical quick response code system, doctor patient diagnosis information sharing system, doctor patient communication system, process, and methods of use that allow a doctor to edit treatment and provide access to modified treatment after patient feedback.

Yet another object of the disclosure is to provide a patient doctor interaction system, medical quick response code system, doctor patient diagnosis information sharing system, doctor patient communication system, process, and methods of use that provides a chat feature. In one embodiment discussed herein, the chat feature with doctors is locked for all patients until a patient unlocks the chat feature using a quick response code (or entering the code manually); Alternatively, the code can constitute numeric or alpha numeric and/or mixed character entry. Furthermore, in this embodiment, the chat quick response code is provided by the provider or doctor during the visit or interaction and can also be included in the rapid response code sheet and/or media.

Another object of the disclosure is to provide a patient doctor interaction system, medical quick response code system, doctor patient diagnosis information sharing system, doctor patient communication system, process, and methods of use that provide each provider and/or doctor with a plurality and/or a set of unique chat quick response codes that can be provided to the practitioner's and/or provider's patient. Furthermore, a unique chat code, and/or a provider specific code, and/or a doctor specific code, may be provided within the same sheet and/or screen that comprise the topic specific QR codes. The patient will then use the unique plurality of and/or set of unique chat quick response codes to activate the chatting feature and/or chat page with the patient's particular doctor only.

Yet another object of the disclosure is to provide a patient doctor interaction system, medical quick response code system, doctor patient diagnosis information sharing system, doctor patient communication system, process, and methods of use that prevent theft and/or unauthorized use of the provider's and/or doctor's chat quick response codes. In one arrangement, as discussed further herein, each provider and/or doctor must pre-activate their own code before providing the quick response code to the patient during the visit and/or encounter and/or medical interview so that only pre-activated codes can be used to activate the chat feature and/or chat page; with this particular provider and/or doctor. In another embodiment, as discussed further herein, the provider and/or doctor can pre-activate the rapid response code by scanning it with the provider's and/or doctor's own device (such as a mobile device or tablet) using the code-activation feature, built-in as part of the patient doctor interaction system, or using a handheld code reader connecting to a computer terminal or by entering the code manually.

Another object of the disclosure is to provide a patient doctor interaction system, medical quick response code system, doctor patient diagnosis information sharing system, doctor patient communication system, process, and methods of use that provides an urgent chat feature for quick patient interaction.

Yet another object of the disclosure is to provide a patient doctor interaction system, medical quick response code system, doctor patient diagnosis information sharing system, doctor patient communication system, process, and methods of use that provides the ability to limit the number of messages that can be sent by a patient after a visit.

Another object of the disclosure is to provide a patient doctor interaction system, medical quick response code system, doctor patient diagnosis information sharing system, doctor patient communication system, process, and methods of use that provides a limitation on urgent chat messages which can be sent.

Yet another object of the disclosure is to provide a patient doctor interaction system, medical quick response code system, doctor patient diagnosis information sharing system, doctor patient communication system, process, and methods of use that provides various forms of post visit education.

Another object of the disclosure is to provide a patient doctor interaction system, medical quick response code system, doctor patient diagnosis information sharing system, doctor patient communication system, process, and methods of use that tracks historical data.

Yet another object of the disclosure is to provide a patient doctor interaction system, medical quick response code system, doctor patient diagnosis information sharing system, doctor patient communication system, process, and methods of use that allows a doctor to save information such as information related to diagnosis.

Another object of the disclosure is to provide a patient doctor interaction system, medical quick response code system, doctor patient diagnosis information sharing system, doctor patient communication system, process, and methods of use that provides alerts.

Yet another object of the disclosure is to provide a patient doctor interaction system, medical quick response code system, doctor patient diagnosis information sharing system, doctor patient communication system, process, and methods of use that provides notifications.

Another object of the disclosure is to provide a patient doctor interaction system, medical quick response code system, doctor patient diagnosis information sharing system, doctor patient communication system, process, and methods of use that can utilize global positioning systems. In one embodiment, a user may utilize the system to find doctors which are nearby and/or vise-versa.

Yet another object of the disclosure is to provide a patient doctor interaction system, medical quick response code system, doctor patient diagnosis information sharing system, doctor patient communication system, process, and methods of use that can utilize global positioning systems to provide localized information.

Another object of the disclosure is to provide a patient doctor interaction system, medical quick response code system, doctor patient diagnosis information sharing system, doctor patient communication system, process, and methods of use that are easy to use.

Yet another object of the disclosure is to provide a patient doctor interaction system, medical quick response code system, doctor patient diagnosis information sharing system, doctor patient communication system, process, and methods of use that are safe to use.

Another object of the disclosure is to provide a patient doctor interaction system, medical quick response code system, doctor patient diagnosis information sharing system, doctor patient communication system, process, and methods of use that are accurate.

Yet another object of the disclosure is to provide a patient doctor interaction system, medical quick response code system, doctor patient diagnosis information sharing system, doctor patient communication system, process, and methods of use that provides a patient interface.

Another object of the disclosure is to provide a patient doctor interaction system, medical quick response code system, doctor patient diagnosis information sharing system, doctor patient communication system, process, and methods of use that provide a physician interface.

Yet another object of the disclosure is to provide a patient doctor interaction system, medical quick response code system, doctor patient diagnosis information sharing system, doctor patient communication system, process, and methods of use that provides a staff interface.

Another object of the disclosure is to provide a patient doctor interaction system, medical quick response code system, doctor patient diagnosis information sharing system, doctor patient communication system, process, and methods of use that can be used with various digital platforms.

Yet another object of the disclosure is to provide a patient doctor interaction system, medical quick response code system, doctor patient diagnosis information sharing system, doctor patient communication system, process, and methods of use that are quick and efficient to use.

Another object of the disclosure is to provide a patient doctor interaction system, medical quick response code system, doctor patient diagnosis information sharing system, doctor patient communication system, process, and methods of use that is easily programmable to various predetermined rule sets.

Yet another object of the disclosure is to provide a patient doctor interaction system, medical quick response code system, doctor patient diagnosis information sharing system, doctor patient communication system, process, and methods of use that are robust.

Another object of the disclosure is to provide a patient doctor interaction system, medical quick response code system, doctor patient diagnosis information sharing system, doctor patient communication system, process, and methods of use that save time for a user.

Yet another object of the disclosure is to provide a patient doctor interaction system, medical quick response code system, doctor patient diagnosis information sharing system, doctor patient communication system, process, and methods of use that save time for a physician.

Another object of the disclosure is to provide a patient doctor interaction system, medical quick response code system, doctor patient diagnosis information sharing system, doctor patient communication system, process, and methods of use that improves upon patient care and patient wellbeing.

Yet another object of the disclosure is to provide a patient doctor interaction system, medical quick response code system, doctor patient diagnosis information sharing system, doctor patient communication system, process, and methods of use that are high quality.

These and other objects, features, or advantages of the present disclosure will become apparent from the specification and claims.

### BRIEF DESCRIPTION OF THE DRAWINGS

FIG. 1. is a diagram illustrating an example of the patient doctor interaction system, medical quick response code system, doctor patient diagnosis information sharing system, doctor patient communication system, process, and methods of use; the diagram showing an example of a patient-healthcare provider interaction for enhancing the success of a healthcare visit by a patient.

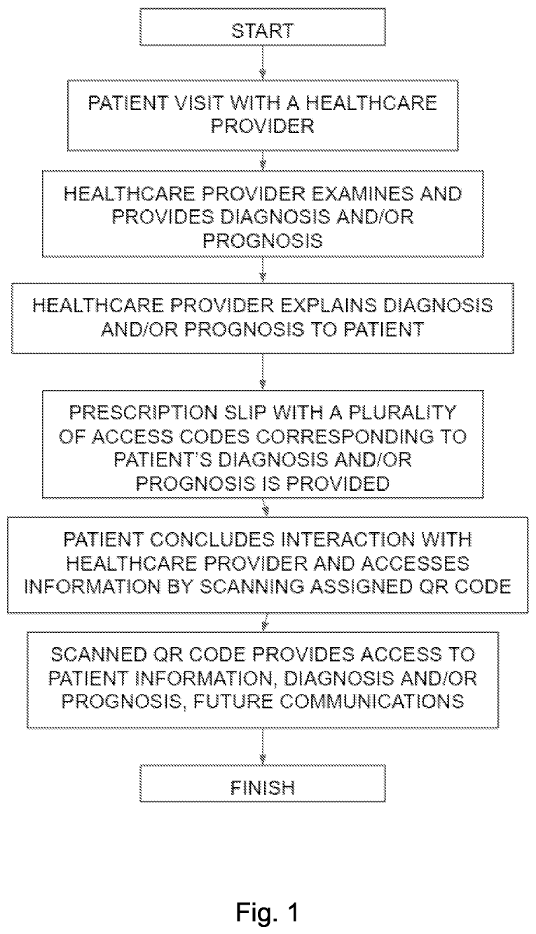

FIG. 2. is a diagram illustrating an example overview of the patient doctor interaction system, medical quick response code system, doctor patient diagnosis information sharing system, doctor patient communication system, process, and methods of use; the diagram illustrating an example of interactions between a healthcare provider, a patient, and the user interface systems.

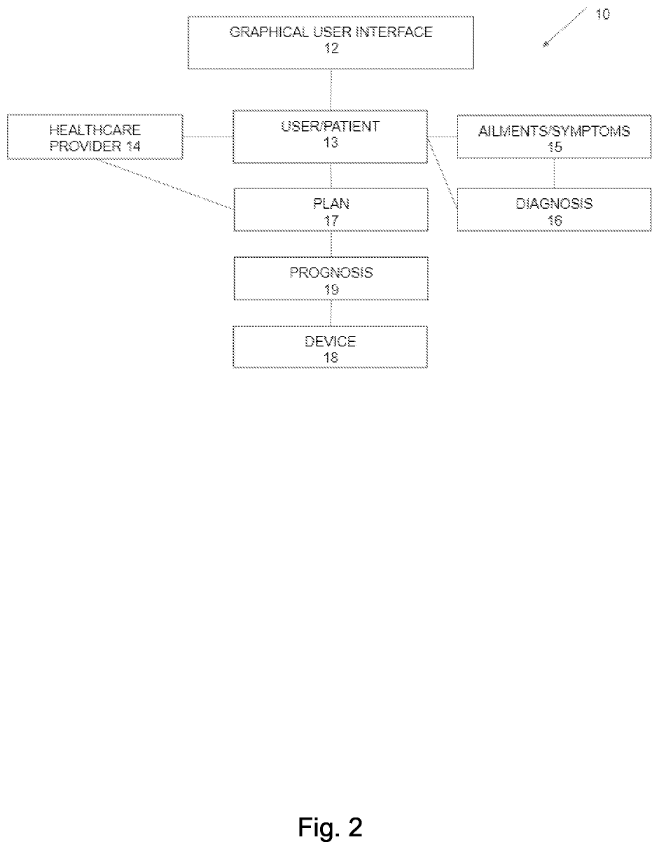

FIG. 3. is a diagram illustrating an example of the patient doctor interaction system, medical quick response code system, doctor patient diagnosis information sharing system, doctor patient communication system, process, and methods of use; the diagram illustrating use of a patient access device which provides access to further interactions between a patient and a healthcare provider after a visit has concluded.

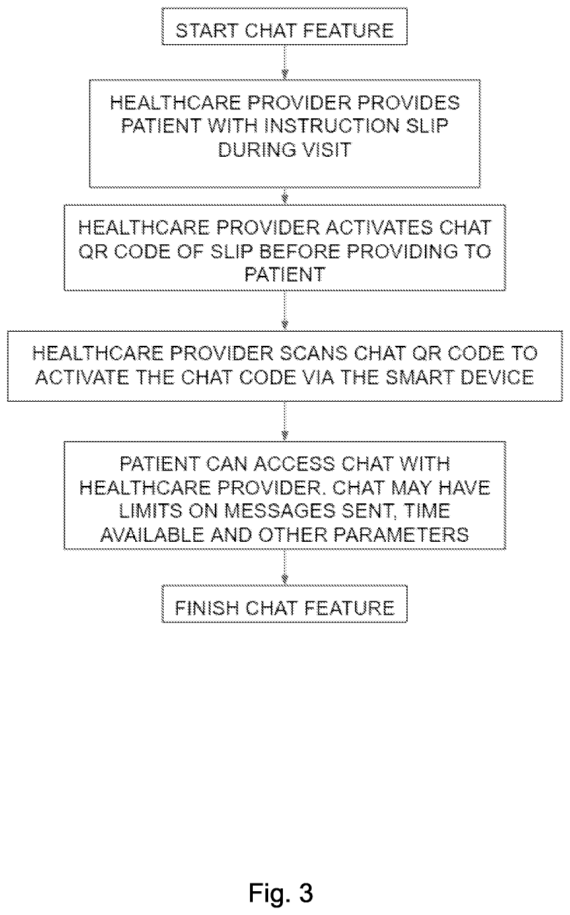

FIG. 4. is a diagram illustrating an example of the patient doctor interaction system, medical quick response code system, doctor patient diagnosis information sharing system, doctor patient communication system, process, and methods of use; the diagram illustrating a prescription slip feature and various features associated with the prescription slip feature.

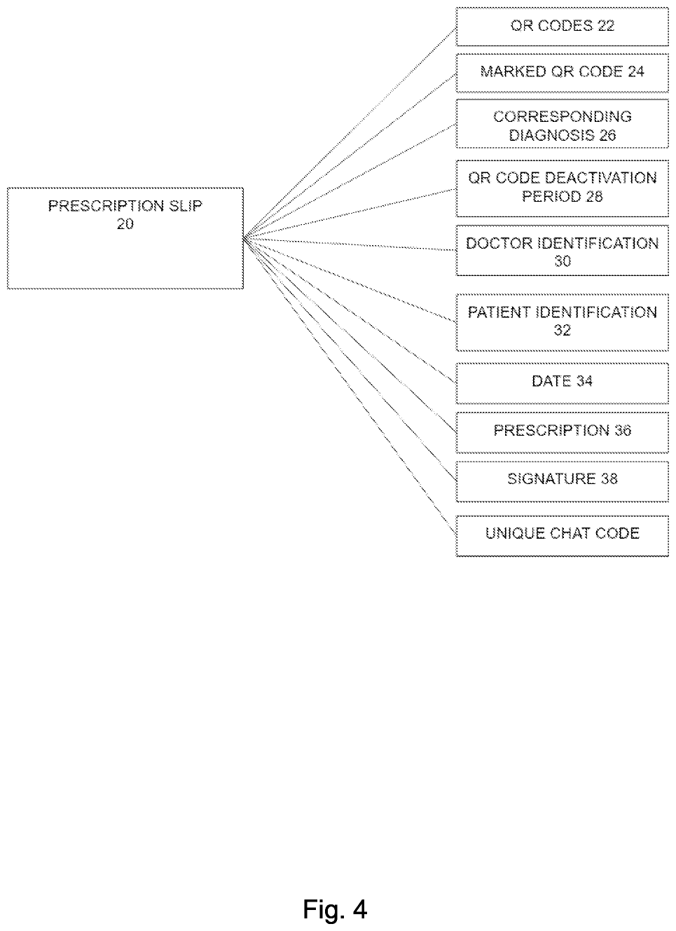

FIG. 5. is a diagram illustrating an example of the patient doctor interaction system, medical quick response code system, doctor patient diagnosis information sharing system, doctor patient communication system, process, and methods of use; the diagram illustrating an instruction slip feature and various features associated with the instruction slip feature.

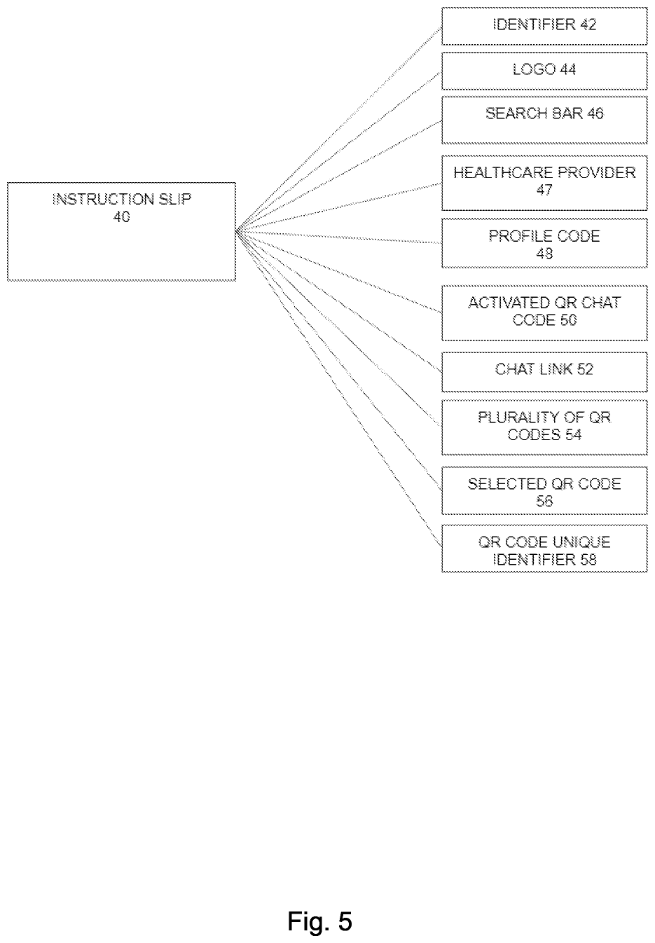

FIG. 6. is a diagram illustrating an example of the patient doctor interaction system, medical quick response code system, doctor patient diagnosis information sharing system, doctor patient communication system, process, and methods of use; the diagram illustrating a user interface and a patient user interface feature; the diagram illustrating various features associated with the patient user interface feature.

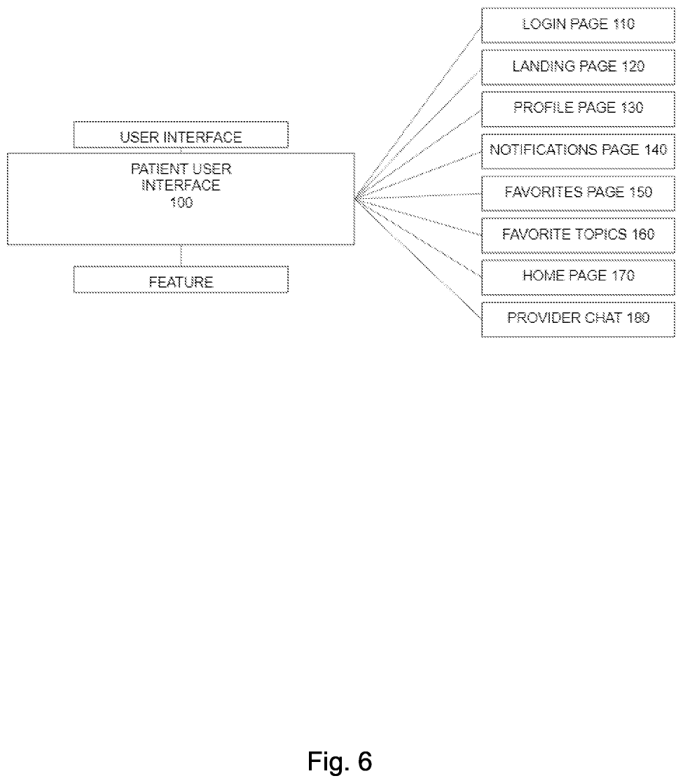

FIG. 7. is a diagram illustrating an example of the patient doctor interaction system, medical quick response code system, doctor patient diagnosis information sharing system, doctor patient communication system, process, and methods of use; the diagram illustrating a user interface and a healthcare provider user interface feature; the diagram illustrating various features associated with the provider user interface feature.

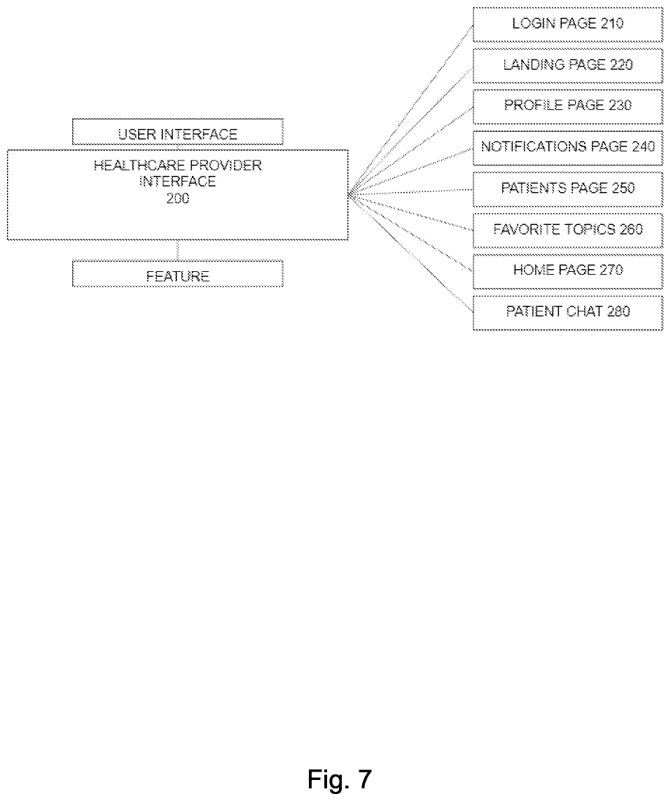

FIG. 8. is an example of an instruction slip of the present embodiment.

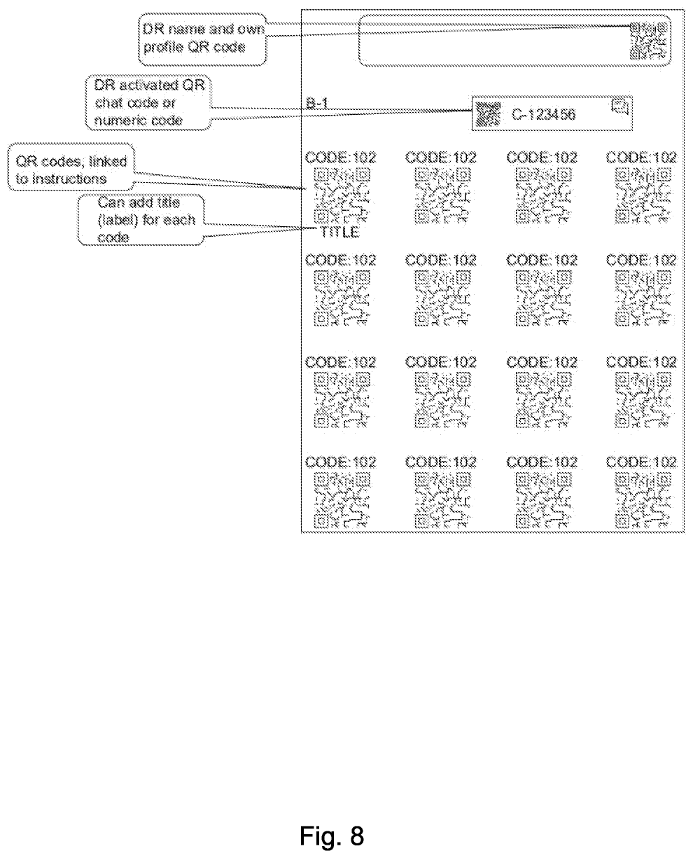

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer removable drive, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code, or virtual code, or framework code suitable for the disclosure herein, or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

In the arrangement shown, as one example, a patient doctor interaction system, medical quick response code system, doctor patient diagnosis information sharing system, doctor patient communication system, process, and methods of use are presented.

### System

With reference to the figures, a patient doctor interaction system, medical quick response code system, doctor patient diagnosis information sharing system, doctor patient communication system, process, and methods of use 10 (hereafter referred to as “doctor patient interaction system”, “medical interview system”, “patient care system”, “physician patient system”, or simply “system”) is formed of any suitable size, shape and design.

In the arrangement shown, as one example, system 10 is configured to improve and enhance physician-patient care by providing novel means and improving above the state of the art in various ways, including, but not limited to, providing information sharing between a patient and a doctor related to a diagnosis and/or treatment plan. More specifically, and without limitation, system 10 improves upon doctor-patient interactions by providing a doctor patient communication system, quick sharing and/or identification system.

In the arrangement shown, as one example, system 10 is configured to include a medical quick response code portal system for use between a practitioner and a client. In the arrangement shown, as one example, system 10 also includes a particular process for sharing information and a method of using system 10.

Furthermore, system 10 is configured to provide the state of the art with and includes as part of the system, a quick response code display. The quick response display may be on a sheet of paper, a smartphone, smart device, tablet, smart watch, or computer, or other display screen such as a computer screen or television or the like (to be further discussed herein). In the arrangement shown, as one example, system 10 includes the quick response code display that shows a plurality of quick response codes for the purpose of scanning these quick response codes.

In the arrangement shown as one example, system 10 is configured to utilize each of the plurality of quick codes as representative of and relating to a discrete patient conditions and/or patient ailment and/or patient treatment. Furthermore, each of the plurality of quick response codes may relate to a customized and particular treatment or the like which is a particular physician's plan for a patient, a particular physician group's plan for a patient, or a particular hospital's plan for a patient. Furthermore, each of the plurality of quick response codes contains information for web addresses and other information related to a landing page containing patient information and/or instructions for a particular condition and/or treatment.

In the arrangement shown, as one example, system 10 is also configured to enhance a clinical visit with a patient (via in-person, audio, video, or a combination thereof), a physician will identify one or more quick response codes of the plurality of quick response codes within the quick response code display. In this way, the quick response code identified by the physician will be the relevant code to and/or appropriate to the particular patient's condition and/or treatment.

In the arrangement shown, as one example, the physician (to be further discussed herein) will have the patient scan the relevant quick code or quick codes with a quick response reader and/or quick response scanner on the patient's device (such as a mobile device or tablet). By scanning the selected quick code or quick codes, a landing page containing patient information is loaded to the device of the patient. The patient can then refer to this page even after the interaction with the physician is complete. This provides the patient with additional and ongoing availability of information. This enables the patient to stay informed and to be able to continue effective treatment without reliance on memory and the like. Said another way, this provides the patient with ongoing access to instructions appropriate for the patient for the particular condition and/or particular treatments needed as directed by the treating doctor. Furthermore, this ongoing access to instructions is unique to the patient because the ongoing information and further consultation is provided, and can be easily updated through the user interface, by the patient's treating doctor and is specific for whatever the patient's circumstances are. In this way, system 10 provides the state of the art, and patients, with a unique personalized and trusted source of health information—that health information pertaining directly to the patient and their unique circumstances.

In the arrangement shown, as one example, system 10 is configured to include the medical quick response code portal system and the medical quick response code method of use also includes back end software which enables the functionality herein as well as graphical user interfaces for both the physician and the patient which enable these various interactions. Furthermore, this system enables staff of the physician and others to customize the information and/or customize the instructions for a particular patient, and/or a particular treatment and/or particular conditions.

Furthermore, system 10 also includes a paper option. In this embodiment the quick response code display may be a prescription booklet and/or printed so as to display the plurality of quick response codes on one or more pages of a booklet. In this way, system 10 also provides a means of providing this plurality of quick response codes to a user through electronic delivery such as through email or text message and the like. In this way, system 10 provides the ability for the quick response codes to be delivered to a patient and/or patients without needing to see the patient in person. In this way, system 10 is configured to provide a meeting between a physician and a patient virtually, a diagnosis to be provided virtually, and the various quick code responses to be provided in a unique way which suits the patient as well as are customized for the patient.

In the arrangement shown, as one example, system 10 also includes, in the embodiment(s) depicted, a graphical user interface 12, a user and/or patient and/or client 13, a practitioner and/or doctor and/or physician 14, an ailment 15, a diagnosis 16, a plan 17, a device 18, a prognosis 19, a prescription slip 20, an instruction slip 40, a patient user interface 100, a physician user interface 200, a communication and/or control system, an application server, and a mobile computing application, among other components, features, and functions.

In the arrangement shown, as one example, system 10 may also comprise remote servers, databases, application servers, application databases, product databases, mobile applications, and/or computers; all of which in continuity or as separate acts fulfill the functions disclosed herein; among other components and features.

### User (or Plurality Thereof)

In the arrangement shown, as one example, system 10 includes a user 13. User 13 (or “patient”, or “guardian”, or “client”) may be any user interacting with or utilizing the system 10. This may include viewing, controlling, analyzing, manipulating, and/or interacting with system 10. Patient 13 is often the patient 13 experiencing the ailment (to be discussed further herein). Furthermore, in some circumstances, it may be a patient's guardian or another who interacts with system 10. Users are not limited to a single user but may be a plurality of users. User may also be a term, herein, which refers to a doctor or health care provider or consultant (to be further discussed herein).

### Provider

In the arrangement shown, as one example, system 10 includes a provider 14. Provider 14 (or “physician”, or “staff”, or “health care provider”, or “consultant”, or “practitioner”) may be any consultant, practitioner, health care provider, doctor, physician, or the like providing services to a client and/or patient. This also may be any doctor or staff member interacting with system 10. Interactions with system 10 may include viewing, controlling, analyzing, manipulating, and/or interacting with system 10. Provider 14 is often the physician analyzing the patient, providing a diagnosis, and providing a treatment plan for the patient.

### Prescription Slip/Instruction Slip

In the arrangement shown, as one example, system 10 includes a prescription slip 20. Prescription slip 20 is formed of any suitable size, shape, and design and is configured to provide a prescription from a doctor to a patient. The prescription slip, or instruction slip is displayed further in the figures herein.

In the arrangement shown, as one example, the prescription slip or the instruction slip may be paper or may be electronic in media and nature. The instruction slip or prescription slip may include an identifier, a logo, a search bar, a doctor name or identification, a profile code, an activated quick response chat code, a chat link, a plurality of quick response codes, a selected quick response code, a quick response code identifier, among other components and features. In one arrangement, and as one example, the quick response codes provided are healthcare provider specific, and in some cases patient specific. The unique quick response (QR) code provides a means for a healthcare provider to edit, modify, manage, and the like their own QR-linked codes and QR-linked instruction pages, and/or QR-linked prescription pages, and/or QR-linked chat pages, and the like. Furthermore, even after information has been provided to a user and/or patient, the information can be changed, updated, and/or edited through an edit feature such that a provider can edit the QR code-linked information using the platform disclosed herein.

### Graphical User Interface

In the arrangement shown, as one example, system 10 may include a graphical user interface 12. Graphical user interface 12 is formed of any suitable size shape and design and is configured to allow a user to view interact with, manipulate, and visually access environmental and system data and information, information related thereto, and/or view various data for various ailments and/or doctors offices and/or patient information and/or add information to and/or change the settings of the system operation.

Graphical user interface 12 is an exemplary method by which systems of the present disclosure may be utilized to improve upon the patient-doctor interactions commonly known to society. Employing graphical user interface 12, enhances a user's interactions with system 10 in the form of awareness and knowledge of data within the system 10. System 10 provides direct links to a user's history, settings, programming, and other information related to a particular office and/or a particular patient and/or a particular client and/or a particular ailment. In this way graphical user interface 12 provides for a means for a user to make adjustments to the operation and functionality of system 10. Additionally, local geographic information with relation to the system can be added, viewed, or retrieved.

Display of Graphical User Interface: In the arrangement shown, as one example, system 10 includes a graphical user interface 12. Graphical user interface 12 may include a display, which is configured to show and display information, including data, for review and interpretation by a user or plurality of users, or a plurality of users interacting with one another. In the arrangement shown, as one example, interactive user display is formed of a display screen, such as that of a desktop computer, laptop computer, monitor, tablet, smart phone, smart TV, projector, virtual reality display or any other device or form of a display.

In the arrangement shown, as one example, interactive user display comprises a series of interactive user display pages, however, the interactive user display may consist of a single page or any other method of displaying information on a display as could be adapted to various size screens, devices, or user preferences. The interactive user display can display various patient and/or medical information which is retrieved and/or requested.

### Patient User Interface

In the arrangement shown, as one example, system 10 includes a patient user interface 100. Patient user interface 100 is formed of any suitable size, shape and design and is configured to provide the primary interactions for the patient with system 10.

In the arrangement shown, as one example, patient user interface 100 includes a plurality of pages, including but not limited to a login page 110—having a logo, a name, an image identifier, a name identifier, a username, a password, a quick response code, a quick response code identifier, and a forgot information feature, among other features and components.

In the arrangement shown, as one example, patient user interface 100 also includes a landing page 120—having a profile page link, a notifications link, a favorite doctors link, a favorite topics link, and a home page link, among other features and components.

In the arrangement shown, as one example, patient user interface 100 also includes a profile page 130—having a name, an identifier, an address, and a plurality of settings, among other features and components.

In the arrangement shown, as one example, patient user interface 100 also includes a notifications page 140—having a page identifier, a favorite topic recall feature, a plurality of notifications, and a notifications link also having a notification indicator, among other features and components.

In the arrangement shown, as one example, patient user interface 100 also includes a favorite doctors page 150—having a plurality of physicians, a number of visits, various doctor information, and a communication link, among other features and components.

In the arrangement shown, as one example, patient user interface 100 also includes a favorite topics page 160—having a search topic feature, a share topic feature, a plurality of topics, a filter topics feature, a topic category feature, an ask about topic link, and a plurality of hyperlinks related to the topic, among other components and features.

In the arrangement shown, as one example, patient user interface 100 also includes a home page 170. Home page 170 may also be referred to as an illness page or ailment page. Home page 170 includes an ailment identifier, a plurality of ailment information, and may also include various hyperlinks related to ailment information, various statistics, and instructions, among other components and features.

In the arrangement shown, as one example, patient user interface 100 also includes a physician chat page 180—having an urgent communication feature, a message limit notice, a patient identifier, a doctor identifier, a plurality of patient messages (once sent), a plurality of doctor message (once sent), and an enter code feature, among other components and features.

Chat Page: In the arrangement shown, as one example, system 10 includes a unique chat feature page 180. In the arrangement shown, as one example, chat page 180 is formed of any suitable size, shape, and design and is configured to provide a unique interaction between the provider and the patient. In the arrangement shown, as one example, the provider or physician can provide the patient with a quick response code. The quick response code may be provided as part of a prescription slip or the like. In this way, the patient can then use the quick response code to gain access to a patient-doctor chat. This allows the patient to interact with the doctor in a unique way, through the scanning of the quick response code. In other words, the patient activates the chat page by scanning the quick response code and/or manually entering the code.

In the arrangement shown, as one example, the patient chat page is locked and/or restricted from use by a patient (as a default setting). However, other settings may be configured as desired by a user, administrator, or the like. Furthermore, and in this way, patients obtain access to the chat page by obtaining or using a chat quick response code. This code may be numbers and letters which are manually entered and/or by scanning a quick response code and/or password or the like.

In the arrangement shown, as one example, once the quick response code, which provides access to the chat page, has been entered and/or scanned by the patient, the chat screen is activated and/or the restriction is removed. Furthermore, once a patient scans the quick response code or activates the chat page, the chat page of the doctor user interface is also activated. In the arrangement shown, as one example, the physician or physician's office, whose chat page is activated, is the same page of the physician who appears on the prescription slip. In this way, and as one example, the patient will know who they are chatting with. Furthermore, other arrangements are also hereby contemplated for use. Other arrangements may include alternative doctor activations such as general practitioners, specialists, a referral practitioner, quick responding nurses on call, or the like. In any case, each doctor will have a unique quick code response identifier so the user can confirm who they are chatting with.

Furthermore, and in the arrangement shown as one example, the quick response code provided to the patient, which triggers and/or activates the doctor-patient chat page will not work unless the code is an active code. In other words, the practitioner or doctor must activate the code before a patient is able to access the quick chat page. In this way, a doctor may have many active codes and inactive codes that can be provided to a patient at any one time, via a prescription slip, or the like. A doctor, in this way, can determine what codes a patient has access to, thus managing the care more critically and providing particular access to particular services and/or information.

Furthermore, in the arrangement shown, as one example, each sheet a doctor provides to a patient has different codes. This feature provides security for health information, security in data delivery and access, security and comfort for physicians that patients have the correct care they need, safety for patients in gathering care and information, and security for doctors knowing that the correct information is being provided to a patient. This also prevents information from being leaked or stolen as well as a number of other security measures which enhance privacy and the like.

For this reason, and in the arrangement shown as one example, the physician or doctor or staff of the group must activate each code. The activation of the user codes will most likely be done during the patient visit and/or doctor patient interaction. However, this may be done shortly after or if a patient requests additional information, such as if a patient requests access to a chat or has questions during ongoing patient care providing. In any sense, once the code is activated by the physician, the patient will have access to this code. In another embodiment, a doctor may restrict time that a patient has access to information, a doctor may also restrict to a quantity limit, such as a quantity of messages on the message page, and the like.

Furthermore, while a patient can activate a chat page by scanning, a doctor can also activate a chat page by scanning. In many circumstances a doctor may wish to provide information to a patient before, after, or even during a medical interview. In this way, a doctor can activate a chat page to provide additional information to the patient quickly and directly. Furthermore, a doctor may desire to check in on a patient and can activate the chat page through the physician user interface. This activation can also be done through scanning and/or manual entry.

### Physician User Interface

In the arrangement shown, as one example, system 10 includes a physician user interface 200. Physician user interface 200 is formed of any suitable size, shape and design and is configured to provide the primary interactions for the physician with system 10.

In the arrangement shown, as one example, physician user interface 200 includes a plurality of pages, including but not limited to a login page 210—having a logo, a name, an image identifier, a name identifier, a username, a password, a quick response code, a quick response code identifier, and a forgot information feature, among other features and components.

In the arrangement shown, as one example, physician user interface 200 also includes a landing page 220—having a profile page link, a notifications link, a patient page, a favorite topics page, and a home page link, among other features and components.

In the arrangement shown, as one example, physician user interface 200 also includes a profile page 230—having a name, an identifier, an address, and a plurality of settings, among other features and components.

In the arrangement shown, as one example, physician user interface 200 also includes a notifications page 240—having a page identifier, a favorite topic recall feature, a plurality of notifications, and a notifications link also having a notification indicator, among other features and components.

In the arrangement shown, as one example, physician user interface 200 also includes a favorite doctors page 250—having a plurality of physicians, a number of visits, various doctor information, and a communication link, among other features and components.

In the arrangement shown, as one example, physician user interface 200 also includes a favorite topics page 260—having a search topic feature, a share topic feature, a plurality of topics, a filter topics feature, a topic category feature, an ask about topic link, and a plurality of hyperlinks related to the topic, among other components and features.

In the arrangement shown, as one example, physician user interface 200 also includes a home page 270. Home page 270 may also be referred to as an illness page or ailment page. Home page 270 includes an ailment identifier, a plurality of ailment information, and may also include various hyperlinks related to ailment information, various statistics, and instructions, among other components and features.

In the arrangement shown, as one example, physician user interface 200 also includes a physician chat page 280—having an urgent communication feature, a message limit notice, a physician identifier, a doctor identifier, a plurality of physician messages (once sent), a plurality of doctor message (once sent), and an enter code feature, among other components and features.

### Computing Platform

In the arrangement shown, as one example, system 10 includes a computing platform (or “computer”, or “computer platform”). Computing platform is formed of any suitable size, shape, and design and is configured to provide computing support, power, and computing processing for both onboard computing functionality as well as communication for off-board or server computing functionality. In this way, an onboard computing system, among other components and features on top of the platform.

In the arrangement shown, as one example, system 10 includes a computer. Computers are formed of any suitable size, shape, and design and are configured to provide for the main off-board computing processing and implementation of computer handling of data from data gathering.

### Remote Computing System

In one arrangement, as is shown, system 10 includes a remote computing system (or “remote computing device”). Remote computing devices are formed of any suitable size, shape, and design and configured to handle onboard computing operations of system 10. Remote computing device is connected with electronic network and/or database and/or server or cloud via communication means and includes a processor, a memory, a microcontroller, a printed circuit board, a microprocessor, a receiver/transceiver, may include at least one antenna, a power supply, and a communications system, among other components.

Computing devices may be formed of any computing device capable of displaying and manipulating data in the manners described herein. Computing devices may include for example a desktop computer, a laptop computer, a tablet, smart phone, or any other computing device or other interactive device.

Computing devices may be a single consolidated component, or alternatively, computing devices may be formed of a plurality of interconnected components that may be co-located or located at different geographic locations. Computing devices may be cloud based or it may be hardware based, or cloud capable. In addition, the connected components of computing devices, including processor, memory, software and interactive user display, may be co-located with computing devices or located at different geographic locations. That is, computing devices may be made of any form of a device or system that individually or collectively performs the computing operations of system 10.

### Application Server

In the arrangement shown, as one example, system 10 comprises remote servers, databases, and/or computers that fulfill the functions disclosed and described herein. In the embodiment depicted, system 10 comprises an application server. Application server comprises one or more computer systems adapted to transmit and receive data regarding selected datasets related to various users and/or datasets related to multiple users. Application server is adapted to query databases with unique identification codes for doctor patient health system 10.

Application server may transmit environmental data and/or surface related documents with respect to a single patient and/or ailment and/or prognosis and/or treatment plan. Application server is also adapted to query a database. This query includes receiving and sending patient and doctor identification and the like. Additionally, application servers may communicate with a cloud computing system or a mobile application, which can also be adapted to present the data in a form conducive to being viewed on a mobile device and/or handheld device.

As one of ordinary skills in the art may understand, application server, database, and other databases mentioned herein may be implemented in one or more servers. Furthermore, each may be on multiple servers to increase system efficiency, especially when handling large data gathering, following extended guidelines, extended ranges, ranges discussed herein. Additionally, multiple servers may have mirrored data to prevent data loss in case of disk failure and/or to decrease access and response times for database queries. In alternative embodiments, application server, and other database procedures may be carried out on computer-readable instructions and data stored on the customer's mobile computing device.

In addition to the above identified features, options, controls, and components, system 10 may also include other features and functionalities, among other options, controls, and components.

### In Operation

Various methods of use are hereby contemplated for use, and are disclosed herein and implemented in accordance with the present disclosure and accompanying figures and materials.

It will be appreciated by those skilled in the art that other various modifications could be made to the system, process, and method of use without parting from the spirit and scope of this disclosure. All such modifications and changes fall within the scope of the claims and are intended to be covered thereby.

What is claimed:

1. A physician patient interaction system, comprising:
a graphical user interface;
a communication system having a process, a memory, and program;
a patient;
a healthcare provider;
an ailment;
the ailment having a plurality of symptoms;
the ailment having a prognosis;
a diagnosis;
a smart device;
a instruction slip;
wherein the instruction slip is provided by the healthcare provider to the patient at the time of an initial visit;
the instruction slip having a plurality of quick response codes;
wherein the patient scans the quick response code designated by the healthcare provider for a information related to the patient;
wherein by scanning the quick response code designated by the healthcare provider, the patient is provided with information related to the patient's ailment and the plan for treating the ailment;
a patient interface;
wherein the patient access information related to the instruction slip through the patient interface;
a healthcare provider interface;
wherein the healthcare provider and the patient can have additional, secure communication after the initial visit has concluded.

2. The system of claim 1, further comprising:
the instruction slip providing a written plan for treating the ailment;
wherein the patient scans the title of each linked topic, wherein the linked topic is linked to the quick response code;
wherein the patient scans the quick response code designated by the healthcare provider for a set of healthcare information, wherein the set of healthcare information includes a plan to treat the patient.

3. The system of claim 1, further comprising:
a prescription slip;
the prescription slip having an order for a use of medicine;

4. The system of claim 1, further comprising:
the instruction slip having an order for treatment.

5. The system of claim 1, further comprising:
wherein the instruction slip is provided electronically.

6. The system of claim 1, further comprising:
wherein a subsequent instruction slip is provided by the healthcare provider to the patient.

7. The system of claim 1, further comprising:
wherein the quick response code also provides a link to written directions for use of medicine, prescribed by the healthcare provider.

8. The system of claim 1, further comprising:
the patient interface having a profile creation page, a login page, a profile page, a notifications page, a favorite healthcare providers page, a favorite topics feature, a home page feature, and a chat with healthcare provider feature.

9. The system of claim 1, further comprising:
the patient interface having a chat feature;
wherein the chat feature is accessible by the patient by scanning a quick response code provided by the healthcare provider;
wherein the chat feature is available to the patient for a limited time, wherein the limited time is a limited number of messages the patient can send to the healthcare provider;
wherein each instruction slip has a unique chat code;
wherein the chat feature is healthcare provider specific; wherein the unique chat code is used to gain access to a chat with a specific healthcare provider;
wherein the chat requires pre-activation by the healthcare provider.

10. The system of claim 1, further comprising:
the patient interface having a chat feature;
wherein the chat feature is accessible by the patient by manually entering an alphanumeric code provided by the healthcare provider;
wherein the chat feature is available to the patient for a limited time, wherein the limited time is a limited time period the patient is able to send communications to the healthcare provider.

11. The system of claim 1, further comprising:
the healthcare provider interface having a profile creation page, a login page, a profile page, a notifications page, a patient's page listing a plurality of patients of the healthcare provider, a favorite topics feature, a home page feature, and a patient chat feature;
wherein a subsequent instruction slip is provided by the healthcare provider to the patient at a time after the initial visit.

12. A system for providing information to patients, comprising:
a graphical user interface;
a communication system having a process, a memory, and program;
a patient;
a healthcare provider;
an ailment;
the ailment having a plurality of symptoms;
the ailment having a prognosis;
a diagnosis;
a smart device;
a instruction slip;
wherein the instruction slip is provided by the healthcare provider to the patient at the time of an initial visit;
the instruction slip having a plurality of quick response codes;
wherein the patient scans the quick response code designated by the healthcare provider for a particular treatment plan, wherein the particular treatment plan includes a plan to treat the patient;
wherein by scanning the quick response code designated by the healthcare provider, the patient is provided with information related to the patient's ailment and the plan for treating the ailment;
a patient interface;
wherein the patient access information related to the instruction slip through the patient interface;
a healthcare provider interface;
wherein the healthcare provider and the patient can have additional, secure communication after the initial visit has concluded;
wherein the communication system operates under a predetermined set of rules at a central processing server;
wherein the communication system receives a permission statement from the healthcare provider allowing communication between the healthcare provider and the patient;
wherein the communication system receives a first message from the patient and transmits the first message to the healthcare provider;
wherein the first message and subsequent messages are coded and routed to and from the healthcare provider and the patient.

13. The system of claim 12, further comprising:
the instruction slip providing a plan for treating the ailment;
wherein a subsequent instruction slip is provided by the healthcare provider to the patient;
wherein the quick response code also provides a link to directions for use of medicine, prescribed by the healthcare provider.

14. The system of claim 12, further comprising:
the instruction slip having an order for use of medicine.

15. The system of claim 12, further comprising:
wherein the instruction slip is provided electronically.

16. The system of claim 12, further comprising:
the patient interface having a profile creation page, a login page, a profile page, a notifications page, a favorite healthcare providers page, a favorite topics feature, a home page feature, and a chat with healthcare provider feature.

17. The system of claim 12, further comprising:
the patient interface having a chat feature;
wherein the chat feature is accessible by the patient by scanning a quick response code provided by the healthcare provider;
wherein the chat feature is available to the patient for a limited time, wherein the limited time is a limited number of messages the patient can send to the healthcare provider.

18. The system of claim 12, further comprising:
the patient interface having a chat feature;
wherein the chat feature is accessible by the patient by manually entering an alphanumeric code provided by the healthcare provider;
wherein the chat feature is available to the patient for a limited time, wherein the limited time is a limited time period the patient is able to send communications to the healthcare provider following the initial visit.

19. The system of claim 12, further comprising:
the healthcare provider interface having a profile creation page, a login page, a profile page, a notifications page, a patient's page listing a plurality of patients of the healthcare provider, a favorite topics feature, a home page feature, and a patient chat feature.

20. A method for providing treatment and ongoing services by a healthcare provider for a patient, comprising the steps:
providing a communication system having a process, a memory, a program, and a graphical user interface;
visiting a healthcare provider by a patient;
diagnosing the patient;
providing the patient with a quick response code associated with a diagnosis;
providing a instruction slip; wherein the instruction slip is provided by the healthcare provider to the patient at the time of an initial visit;
scanning the quick response code; wherein the patient scans the quick response code designated by the healthcare provider for a particular treatment plan, wherein the particular treatment plan includes a plan to treat the patient;
accessing information associated with the diagnosis;
accessing the communication platform;
sending a first message from the patient to the healthcare provider;
communicating about a visit;
providing additional information and additional communication between the healthcare provider and the patient.
